(19) United States
(12) Patent Application Publication
(10) Pub. No.: US 2006/0257350 A1
(43) Pub. Date: Nov. 16, 2006

(54) Shave composition containing three types of lubricants

(75) Inventors: Ali Alwattari, Braintree, MA (US); Ronald Patrick McLaughlin, Medford, MA (US)

Correspondence Address: THE PROCTOR & GAMGLE COMPANY;INTELLECTUAL PROPERTY DIVISION, WINTON HILL TECHNICAL CENTER - BOX 161, 6110 CENTER HILL AVENUE, CINCINATTI, OH, 45224, US

(73) Assignee: The Gillette Company

(21) Appl. No.: 11/129,694

(22) Filed: May 13, 2005

(51) Int. Cl. A61K 8/81

(52) U.S. Cl. 424/73

(57) ABSTRACT

Disclosed is a shaving composition, preferably a shaving composition in the form of a post-foaming gel, that includes a combination of three types of lubricants. The three lubricants are selected from: (a) a lubricious water soluble polymer; (b) water insoluble particles; and (c) a hydrogel-forming (or water swellable) polymer. In particular the shaving composition comprises, in percent by weight, about 60% to about 93%, preferably about 70% to about 85%, water, about 2% to about 25%, preferably about 5% to about 20%, water dispersible (or soluble) surface active agent, about 0.005% to about 2%, preferably about 0.01% to about 0.5%, lubricious water soluble polymer, about 0.01% to about 3%, preferably about 0.1% to about 1%, water insoluble particles, and about 0.0005% to about 3%, preferably about 0.001% to about 0.5%, hydrogel-forming polymer. When the composition is in the form of a post-foaming shave gel, then it will also include about 1% to about 6%, preferably about 2% to about 5%, volatile post-foaming agent.

### BACKGROUND OF THE INVENTION

The present invention relates to a shaving composition, preferably one in the form of a post-foaming gel, that contains three types of lubricants.

Currently, a widely used form of shaving preparation is the type referred to as a post-foaming shave gel. These post-foaming shave gels are now well-known and have been described, for example, in U.S. Pat. Nos. 2,995,521 (Bluard), 3,541,581 (Monson), 4,405,489 (Sisbarro), 4,528,111 (Su), 4,651,503 (Anderson), 5,248,495 (Patterson), 5,308,643 (Osipow), 5,326,556 (Barnet), and 5,500,211 (George). Such compositions generally take the form of an oil-in-water emulsion in which the post-foaming agent, generally a volatile (i.e., low boiling point) aliphatic hydrocarbon, is solubilized in the oil phase, and the water phase comprises a water-dispersible soap or interrupted soap component. The product is generally packaged in an aerosol container with a barrier, such as a piston or collapsible bag, to separate the post-foaming gel from the propellant required for expulsion of the product. The product is dispensed as a clear, translucent or opaque gel that is substantially free from foaming until it is spread over the skin, at which time it produces a foam lather generated by the volatilization of the volatile hydrocarbon foaming agent.

Various attempts have been made to increase the lubricity of shaving compositions. For example, in some cases a lubricious water soluble polymer such as polyethylene oxide or polyvinylpyrrolidone has been added. See, for example, U.S. Pat. Nos. 5,560,859 (Hartmann), 5,858,343 (Szymczak) and WO 02/087519 (Dodd). In other cases, water insoluble particles have been added, including water insoluble polymer particles, such as polytetrafluoroethylene, polyethylene, or polyamide (nylon) particles, and water insoluble inorganic particles such as titanium dioxide or glass beads. See, for example, U.S. Pat. Nos. 5,587,156, 4,155,870 and EP 0 829 259.

While each of the various proposed additives provides some increase in lubricity, there is still a need to improve the lubricity of shaving compositions to a greater extent. Unfortunately, one cannot simply add more of one type of additive to increase lubricity because increased levels can adversely affect desired attributes of the composition or may be too costly or may cause formulation or manufacturing problems. For example, polyethylene oxide can cause stringiness, while insoluble particulates like polytetrafluoroethylene can degrade aesthetic attributes.

It is an object of the present invention to provide a shaving composition, particularly a composition in the form of a post-foaming shave gel, with improved lubricity by including a particular combination of lubricious additives.

### SUMMARY OF THE INVENTION

The present invention embraces a shaving composition, preferably a shaving composition in the form of a post-foaming gel, that includes a combination of three types of lubricants. The three lubricants are selected from: (a) a lubricious water soluble polymer; (b) water insoluble particles; and (c) a hydrogel-forming (or water swellable) polymer. In particular the shaving composition comprises, in percent by weight, about 60% to about 93%, preferably about 70% to about 85%, water, about 2% to about 25%, preferably about 5% to about 20%, water dispersible (or soluble) surface active agent, about 0.005% to about 2%, preferably about 0.01% to about 0.5%, lubricious water soluble polymer, about 0.01% to about 3%, preferably about 0.1% to about 1%, water insoluble particles, and about 0.0005% to about 3%, preferably about 0.001% to about 0.5%, hydrogel-forming polymer. When the composition is in the form of a post-foaming shave gel, then it will also include about 1% to about 6%, preferably about 2% to about 5%, volatile post-foaming agent.

### DETAILED DESCRIPTION OF THE INVENTION

The term “fatty”, as used herein, means a hydrocarbon chain having 12-22 carbon atoms (C₁₂₋₂₂), preferably 14-18 carbon atoms (C₁₄₋₁₈). The chain may be straight or branched and may be saturated or unsaturated (typically one or two double bonds in the chain). The term “water dispersible”, as used herein, means that a substance is either substantially dispersible or soluble in water.

The shaving composition of the present invention comprises, in percent by weight, about 60% to about 93%, preferably about 70% to about 85%, water, about 2% to about 25%, preferably about 5% to about 20%, water dispersible surface active agent, about 0.005% to about 3%, preferably about 0.01% to about 1%, lubricious water soluble polymer, about 0.01% to about 5%, preferably about 0.1% to about 2%, water insoluble particles, and about 0.0005% to about 3%, preferably about 0.001% to about 0.5%, hydrogel-forming polymer. Preferably, the composition is in the form of a post-foaming shave gel and will additionally include about 1% to about 6%, preferably about 2% to about 5%, volatile post-foaming agent.

The water dispersible surface active agent is preferably one that is capable of forming a lather and may comprise a soap, an interrupted soap, a detergent, an anionic surfactant, a non-ionic surfactant or a mixture of one or more of these. The soaps include, for example, the sodium, potassium and lower alkanolamine (preferably triethanolamine) salts of C₁₂₋₂₂, preferably C₁₄₋₁₈, fatty acids. Typical fatty acids include lauric, myristic, palmitic and stearic acid and mixtures thereof. The preferred fatty acids are palmitic and stearic. The interrupted soaps include, for example, the sodium, potassium and lower alkanolamine (preferably triethanolamine) salts of N-fatty acyl sarcosines, wherein the fatty acyl moiety has 12 to 22, preferably 14 to 18, carbon atoms. Typical sarcosines include stearoyl sarcosine, myristoyl sarcosine, palmitoyl sarcosine, oleoyl sarcosine, lauroyl sarcosine, cocoyl sarcosine and mixtures thereof. The soaps and the interrupted soaps may be utilized in preneutralized form (i.e., as the sodium, potassium or alkanolamine salt) or in the free acid form followed by subsequent neutralization with sodium hydroxide, potassium hydroxide and/or lower alkanolamine (preferably triethanolamine). In any event, the final composition must contain sufficient base to neutralize or partially neutralize the soap component and adjust the pH to the desired level (typically between 5 and 10, more typically between 6 and 9). It is most preferred that the composition of the present invention includes a soap (e.g., triethanolamine palmitate/stearate) or an interrupted soap (e.g., triethanolamine stearoyl/myristoyl sarcosinate), or a mixture thereof.

The water dispersible surface active agent may also optionally include a non-ionic, amphoteric and/or anionic surfactant. Suitable non-ionic surfactants will typically have an HLB of 9 or more and include the polyoxyethylene ethers of fatty alcohols, acids and amides, particularly those having 10 to 20, preferably 12 to 18, carbon atoms in the fatty moiety and about 2 to 60, preferably 4 to 30, ethylene oxide units. These include, for example, Oleth-20, Steareth-21, Ceteth-20, Laureth-4 and Laureth-23. Other non-ionic surfactants include the polyoxyethylene ethers of alkyl substituted phenols, such as Nonoxynol-4 and Nonoxynol-20, fatty alkanolamides such as Lauramide DEA and Cocamide MEA, polyethoxylated sorbitan esters of fatty acids, such as Polysorbate-20, lauryl polyglucoside, sucrose laurate, and polyglycerol 8-oleate. Suitable amphoteric surfactants include, for example, the betaines and sultaines such as cocoamidopropyl betaine, coco dimethyl carboxymethyl betaine, coco sultaine and the like. Suitable anionic surfactants include, for example, the sodium, potassium, ammonium and substituted ammonium salts (such as the mono-, di- and triethanolamine salts) of C₈-C₂₂, preferably C₁₂-C₁₈, alkyl sulfates (e.g., sodium lauryl sulfate, ammonium lauryl sulfate), alkyl sulfonates (e.g., ammonium lauryl sulfonate), alkylbenzene sulfonates (e.g. ammonium xylene sulfonate), acyl isethionates (e.g. sodium cocoyl isethionate), acyl lactylates (e.g. sodium cocoyl lactylate) and alkyl ether sulfates (e.g., ammonium laureth sulfate). The surface active agent may typically include up to about 10%, preferably 1 to 8%, of non-ionic, amphoteric and/or anionic surfactants.

The post-foaming agent, when included in the shaving composition, may be any volatile hydrocarbon or halohydrocarbon with a sufficiently low boiling point that it will volatilize and foam the gel upon application to the skin, but not so low that it causes the gel to foam prematurely. The typical boiling point of such an agent generally falls within the range of −20° to 40° C. Preferred post-foaming agents are selected from saturated aliphatic hydrocarbons having 4 to 6 carbon atoms, such as n-pentane, isopentane, neopentane, n-butane, isobutane, and mixtures thereof. Most preferred is a mixture of isopentane and isobutane in a weight ratio (IP:IB) of about 1:1 to about 9:1, preferably about 2:1 to about 7:1, most preferably about 3:1. The post-foaming agent will normally be selected so as to provide a vapor pressure at 20° C. of about 3 to about 20 psig, preferably about 5 to about 15 psig. The post-foaming agent will be present in an amount to provide the shaving composition with a sufficiently rapid turnover—that is, transition from gel to foam when contacted with the skin—typically, in about 2 to about 30 seconds, preferably in about 5 to about 15 seconds.

The lubricious water soluble polymer will generally have a molecular weight greater between about 300,000 and 15,000,000 daltons, preferably more than about one million daltons, and will include a sufficient number of hydrophilic moieties or substituents on the polymer chain to render the polymer water soluble. The polymer may be a homopolymer, copolymer or terpolymer. Examples of suitable lubricious water soluble polymers include polyethylene oxide, polyvinylpyrrolidone, and polyacrylamide. A preferred lubricious water soluble polymer comprises polyethylene oxide, and more particularly a polyethylene oxide with a molecular weight of about 1 to about 5 million daltons. Particularly suitable polyethylene oxides include, for example, PEG-23M (MW≅1 million), PEG-45M (MW≅2 million) and PEG-90M (MW≅4 million). The lubricious water soluble polymer will generally be included in the shaving composition in an amount of about 0.005% to about 3%, preferably about 0.01% to about 1%, by weight.

The water insoluble particles may include inorganic particles or organic polymer particles. Examples of inorganic particles include titanium dioxide, silicas, silicates and glass beads, with glass beads being preferred. Examples of organic polymer particles include polytetrafluoroethylene particles, polyethylene particles, polypropylene particles, polyurethane particles, polyamide particles, or mixtures of two or more of such particles. Any of the forgoing particles may also include a surface treatment to make the particles more readily dispersible or improve their cosmetic aesthetics. Preferred are polytetrafluoroethylene particles (e.g., PTFE particles available from MicroPowders, Inc. under the tradename Microslip). Preferably the water insoluble particles will have an average particle size of about 1 μm to about 100 μm, more preferably about 2 μm to about 50 μm, and most preferably about 5 μm to about 15 μm. The particles may be of any desired shape including spherical bead, elongated fiber or irregular shape, with spherical bead being the preferred shape. Generally the water insoluble particles will be included in the shaving composition in an amount of about 0.01% to about 5%, preferably about 0.1% to about 2%, by weight.

The hydrogel-forming polymer is a highly hydrophilic polymer that, in water, forms organized three-dimensional domains of approximately nanometer scale. The hydrogel-forming polymer generally has a molecular weight greater than about one million daltons (although lower molecular weights are possible) and typically is at least partially or lightly crosslinked and may be at least partially water insoluble, but it also includes a sufficient number of hydrophilic moieties so as to enable the polymer to trap or bind a substantial amount of water within the polymer matrix and thereby form three-dimensional domains. It has been found that shave gel compositions that include the hydrogel-forming polymer have improved gel structure and reduced coefficient of friction (i.e., increased lubricity). Examples of suitable hydrogel-forming polymers include a polyacrylic acid or polymethacrylic acid partially esterified with a polyhydric alcohol; hydrophilic polyurethanes; lightly crosslinked polyethylene oxide; lightly crosslinked polyvinyl alcohol; lightly crosslinked polyacrylamide; hydrophobically modified hydroxyalkyl cellulose; hydroxyethyl methacrylate; and crosslinked hyaluronic acid. Generally, the hydrogel-forming polymer will be included in the shaving composition in an amount of about 0.0005% to about 3%, preferably about 0.001% to about 0.5%, more preferably about 0.002% to about 0.1%, by weight.

A preferred hydrogel-forming polymer comprises polyacrylic acid partially esterified (e.g., about 40% to 60%, preferably about 50%, esterified) with glycerin. Such a polymer includes glyceryl acrylate/acrylic acid copolymer (MW>one million). It is believed that the glyceryl acrylate/acrylic acid copolymer forms a clathrate that holds water, which, upon release supplies lubrication and moisturization to the skin. A preferred source of glyceryl acrylate/acrylic acid copolymer is available from ISP Technologies, Inc. (United Guardian Inc.) under the tradename Lubrajel®, particular the form known as Lubrajel® oil which contains about 1.0%-1.3% glyceryl acrylate/acrylic acid copolymer in aqueous glycerin (˜40% glycerin). Lubrajel® oil also includes about 0.6% PVM/MA copolymer (also known as methoxyethylene/maleic anhydride copolymer), which may further contribute to the lubricity of this source. Most preferably, the shaving composition will include about 0.25% to about 4% Lubrajel® oil in order to provide a preferred level of about 0.002% to about 0.05% of the glyceryl acrylate/acrylic acid copolymer. This amount of Lubrajel® oil will also provide about 0.001% to about 0.03% of PVM/MA copolymer.

Although not necessary to forming a useful shave gel composition, other cosmetic ingredients may be advantageously added to improve the application aesthetics and/or achieve other shave benefits. For example, the composition may include one or more of the following components: beard wetting agents, skin conditioning agents (e.g., vitamins A, C and E, aloe, allantoin, panthenol, alpha-hydroxy acids, phospholipids, triglycerides, botanical oils, amino acids), foam boosters, emollients, humectants (e.g., glycerin, sorbitol, propylene glycol), fragrances, colorants, antioxidants, preservatives, etc. It is particularly preferred to include glycerin in the shave gel composition of the present invention, preferably in an amount of about 0.1% to about 3%, more preferably about 0.3% to about 1%, by weight. Glycerin improves the emolliency and lubricity of the composition.

It may be advantageous to include a sorbitan fatty ester or a sucrose fatty ester, typically in an amount of about 0.1% to about 3%, preferably about 0.3% to about 2%, by weight. These materials have multifunctional properties of emulsifier, moisturizer and anti-irritant. Sorbitan fatty esters include sorbitan stearate, sorbitan oleate, sorbitan isostearate, sorbitan laurate, sorbitan dioleate, etc. Sucrose fatty esters include sucrose stearate, sucrose oleate, sucrose isostearate, sucrose cocoate, sucrose distearate, etc. The sorbitan esters and sucrose esters may be mixtures of mono-, di- and tri-esters.

It may also be desirable to include an ester of a fatty acid, typically in an amount of about 0.5% to about 5%, preferably about 1% to about 4%, by weight. Useful fatty esters include glyceryl fatty esters such as, for example, glyceryl oleate and glyceryl dioleate, and fatty alcohol esters such as, for example, isostearyl linoleate, isocetyl oleate, and isostearyl isostearate. These materials provide emolliency, lubrication and gel structure.

It may further be desirable to include a propoxylated fatty amide, typically in an amount of about 0.5% to about 5%, preferably about 1% to about 3%, by weight. The propoxylated fatty amide will typically have from 1 to 3 propoxyl groups attached to a hydroxyloweralkyl fatty amide. Thus, suitable propoxylated fatty amides include, for example, PPG-2-hydroxyethyl coco/isostearamide, PPG-3-hydroxyethyl linoleamide, and PPG-2-hydroxyethyl cocamide.

The shave gel composition may include a water-soluble gelling aid or thickening agent to improve its consistency and stability, as well as to adjust its viscosity. These may include, for example, hydroxyalkyl cellulose polymers such as hydroxyethyl cellulose and hydroxypropyl cellulose (sold under the trademarks “Natrosol” and “Klucel” respectively), PEG-150 distearate, carboxymethyl cellulose, and cellulose methyl ether (sold under the trademark “Methocel”). Other suitable materials include the polysaccharide gums such as, for example, xanthan gum, carrageenan gum, guar gum, locust bean gum, and hydroxypropyl guar gum. The gelling aid or thickening agent is typically included in an amount of about 0.01% to 5%, preferably about 0.1% to 2%, by weight of the composition.

The shaving compositions of the present invention may be formulated as an aerosol foam, a post-foaming gel (which is the preferred form) or a non-aerosol gel or lather. It may be packaged in any suitable dispenser normally used for dispensing shaving compositions. These include collapsible tubes, pump or squeeze containers, and aerosol-type dispensers, particularly those with a barrier to separate the shaving composition from the propellant required for expulsion. The latter type of dispensers include: (1) mechanically pressurized bag-in-sleeve systems in which a thin-walled inner bag containing the product is surrounded by an outer elastic sleeve that is expanded during the product filling process and provides dispensing power to expel the product (e.g., the ATMOS System available commercially from the Exxel Container Co.); (2) manually activated air pump spray devices in which a pump system is integrated into the container to allow the user to pressurize the container with air in order to expel the product (e.g., the “AIRSPRAY” system available from Airspray International); (3) piston barrier systems in which the product is separated from the driving means by a tight-fitting piston which seals to the side of the container and may be driven by a spring under tension, by a vacuum on the product side of the piston, by finger pressure, by gas pressure to the piston, or by a variety of other means known to the packaging industry; and (4) bag-in-can (SEPRO) systems in which the product is contained in a flexible bag within a can, with a suitable propellant injected into the space between the can and the flexible bag. It is preferred to protect the composition from oxidation and heavy metal contamination. This can be achieved, for example, by purging the composition and container with nitrogen to remove oxygen and by utilizing inert containers (e.g., plastic bottles or bags, aluminum cans or polymer coated or lined cans).

The invention may be further described by the following examples in which all parts and percentages are by weight (unless otherwise indicated).

### EXAMPLES 1-5

Post-Foaming Shave Gel

Weight Percent

| Ingredient | Ex. 1 | Ex. 2 | Ex. 3 | Ex. 4 | Ex. 5 |
|---|---|---|---|---|---|
| Water | 78.24 | 75.02 | 75.94 | 75.55 | 75.64 |
| Triethanolamine | 5.88 | 5.88 | 5.88 | 5.88 | 5.88 |
| Palmitic acid | 7.53 | 7.53 | 7.53 | 7.53 | 7.53 |
| Stearic acid | 2.53 | 2.53 | 2.53 | 2.53 | 2.53 |
| Glyceryl Oleate | 1.94 | 1.94 | 1.94 | 1.94 | 1.94 |
| PEG-23M | 0.05 | 0.05 | 0.05 | 0.05 | 0.05 |
| PEG-90M | | 0.06 | 0.06 | 0.06 | 0.06 |
| Hydroxyethylcellulose | 0.49 | 0.49 | 0.49 | 0.49 | 0.49 |
| Lubrajel ® oil* | 0.49 | 0.97 | 0.49 | 0.97 | 0.49 |
| Sorbitol | | 0.97 | 0.97 | 0.97 | 0.97 |
| Glycerin | | 0.49 | | | 0.49 |
| PTFE powder | | 0.15 | 0.15 | 0.15 | 0.15 |
| Fragrance | | 0.87 | 0.78 | 0.78 | 0.78 |
| Colorant | | 0.10 | 0.29 | 0.19 | 0.10 |
| Other (e.g. Vit. E, Aloe, etc.) | | 0.10 | 0.05 | 0.06 | 0.05 |
| Isopentane/isobutane (3:1) | 2.85 | 2.85 | 2.85 | 2.85 | 2.85 |

*Lubrajel oil contains 1.0%-1.3% Glyceryl Acrylate/Acrylic Acid Copolymer (MW ≅ 1 million)

The above-described compositions are made in the following manner: The water soluble polymers (polyethylene oxide, hydroxyethylcellulose) are added to water and mixed until the polymers are completely dissolved (about 30 min.). The aqueous mixture is then heated and the glyceryl oleate, sorbitol and fatty acids are added at about 60° C. and well mixed while the heating continues. At 80-85° C. the triethanolamine is added and mixed for about 20 minutes to form the aqueous soap phase. After cooling the aqueous soap phase to room temperature, the remaining components (i.e., Lubrajel® oil, glycerin, fragrance, colorant, botanicals) are added to the aqueous soap phase and mixed well to form the gel concentrate. (Water may be added if required to bring the batch weight to 100%, thereby compensating for any water loss due to evaporation.) The concentrate is then combined with the volatile post-foaming agent under pressure within the filling line and filled into bottom-gassed aerosol cans with shearing through the valve under nitrogen pressure. (Note: if, instead of Lubrajel® oil, a different hydrogel-forming polymer is utilized, it is preferred to pre-hydrate the polymer in water prior to inclusion in the formulation.)

The above-described shave gel compositions of the present invention provide superior lubrication and moisturization, as well as superior gel strength, compared to similar compositions that do not include the three types of lubricant additives described herein.

While particular embodiments of the invention have been shown and described for illustrative purposes, it will be apparent to those skilled in the art that various changes and modifications may be made without departing from the spirit and scope of the invention, which is defined by the claims which follow.

What is claimed is:

1. A shaving composition comprising water, a water dispersible surface active agent, a lubricious water soluble polymer, water insoluble particles, and a hydrogel-forming polymer.

2. The shaving composition of claim 1 wherein the composition is in the form of a post-foaming gel and additionally comprises a volatile post-foaming agent.

3. The shaving composition of claim 1 wherein the composition comprises, in percent by weight, about 60% to about 93% water, about 2% to about 25% water dispersible surface active agent, about 0.005% to about 3% lubricious water soluble polymer, about 0.01% to about 5% water insoluble particles, and about 0.0005% to about 3% hydrogel-forming polymer.

4. The shaving composition of claim 3 wherein the composition is in the form of a post-foaming gel and additionally comprises about 1% to about 6% volatile post-foaming agent.

5. The shaving composition of claim 1 wherein the composition comprises, in percent by weight, about 70% to about 85% water, about 5% to about 20% water dispersible surface active agent, about 0.01% to about 1% lubricious water soluble polymer, about 0.1% to about 2% water insoluble particles, and about 0.001% to about 0.5% hydrogel-forming polymer.

6. The shaving composition of claim 5 wherein the composition is in the form of a post-foaming gel and additionally comprises about 2% to about 5% volatile post-foaming agent.

7. The shaving composition of claim 1, 2, 3 or 4 wherein the lubricious water soluble polymer is selected from the group consisting of polyethylene oxide, polyvinylpyrrolidone, and mixtures thereof.

8. The shaving composition of claim 1 wherein the water insoluble particles are water insoluble organic polymer particles.

9. The shaving composition of claim 1, 2, 3 or 4 wherein the water insoluble organic polymer particles are selected from the group consisting of polytetrafluoroethylene particles, polyethylene particles, polypropylene particles, polyurethane particles, polyamide particles, and a mixture of two or more of these particles.

10. The shaving composition of claim 1 wherein the hydrogel-forming polymer is selected from the group consisting of a polyacrylic acid or polymethacrylic acid partially esterified with a polyhydric alcohol, a hydrophilic polyurethane, a lightly crosslinked polyethylene oxide, a lightly crosslinked polyvinyl alcohol, a lightly crosslinked polyacrylamide, a hydrophobically modified hydroxyalkyl cellulose, a hydroxyethyl methacrylate, a crosslinked hyaluronic acid, and a mixture of two or more of these polymers.

11. The shaving composition of claim 1, 2, 3 or 4 wherein the hydrogel-forming polymer comprises a polyacrylic acid or polymethacrylic acid partially esterified with a polyhydric alcohol.

12. The shaving composition of claim 1, 2, 3 or 4 wherein the hydrogel-forming polymer comprises a polyacrylic acid partially esterified with glycerin.

13. The shaving composition of claim 1, 2, 3 or 4 wherein the hydrogel-forming polymer comprises glyceryl acrylate/acrylic acid copolymer.

14. The shaving composition of claim 1, 2, 3 or 4 wherein the lubricious water soluble polymer is selected from the group consisting of polyethylene oxide, polyvinylpyrrolidone, and mixtures thereof; the water insoluble organic polymer particles are selected from the group consisting of polytetrafluoroethylene particles, polyethylene particles, polypropylene particles, polyurethane particles, polyamide particles, and a mixture of two or more of these particles; and the hydrogel-forming polymer is selected from the group consisting of a polyacrylic acid or polymethacrylic acid partially esterified with a polyhydric alcohol, a hydrophilic polyurethane, a lightly crosslinked polyethylene oxide, a lightly crosslinked polyvinyl alcohol, a lightly crosslinked polyacrylamide, a hydrophobically modified hydroxyalkyl cellulose, a hydroxyethyl methacrylate, a crosslinked hyaluronic acid, and a mixture of two or more of these polymers.

15. The shaving composition of claim 1, 2, 3 or 4 wherein the lubricious water soluble polymer comprises polyethylene oxide, the water insoluble organic polymer particles comprise polytetrafluoroethylene particles, and the hydrogel-forming polymer comprises a polyacrylic acid or polymethacrylic acid partially esterified with a polyhydric alcohol.

16. The shaving composition of claim 1 wherein the water dispersible surface active agent comprises a soap or an interrupted soap.

17. The shaving composition of claim 14 wherein the water dispersible surface active agent comprises a soap or an interrupted soap.
